(19) United States
(12) Patent Application Publication
(10) Pub. No.: US 2010/0074947 A1
(43) Pub. Date: Mar. 25, 2010

(54) Pharmaceutical Formulations

(76) Inventors: Adrian Brown, Harlow (GB); Wayne M. Matthews, Harlow (GB); Daniel N. Margetson, Harlow (GB); Stephen Mark McAllister, Sandwich (GB); Danielle Genevieve Rebecca Russell, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/483,477

(22) Filed: Jun. 12, 2009

Related U.S. Application Data
(60) Provisional application No. 61/061,275, filed on Jun. 13, 2008.

(51) Int. Cl. A61K 9/48

(52) U.S. Cl. 424/452

(57) ABSTRACT

The present invention is directed to novel pharmaceutically acceptable polymeric compositions suitable for melt extrusion and injection moulding of single or multi-component pharmaceutical dosage forms comprising a plurality of drug substance containing sub-units, being capsule compartments and/or solid sub-units comprising a solid matrix of a polymer which contains a drug substance, the sub-units being connected together in the assembled dosage form.

This application claims the benefit of U.S. Provisional application No. 61/061,275, filed 13 Jun. 2008, which is incorporated herein in its entirety.

### FIELD OF THE INVENTION

This invention relates to the preparation of injection molded single or multi-component dosage forms using novel pharmaceutically acceptable polymeric blends.

### BACKGROUND OF THE INVENTION

Various types of pharmaceutical dosage forms are known for oral dosing. Pharmaceutical capsules are well known, generally being intended for oral dosing. Such capsules generally comprise an envelope wall of a pharmaceutically acceptable, e.g. orally ingestible, polymer material such as gelatin, although other materials for capsule walls, e.g. starch and cellulose based polymers are also known. Such capsules generally have soft walls made by making a film on a capsule former, which is then allowed to dry. Rigid walled capsules made by injection molding are also known, see for example U.S. Pat. Nos. 4,576,284; U.S. Pat. No. 4,591,475; U.S. Pat. No. 4,655,840; U.S. Pat. No. 4,738,724; U.S. Pat. No. 4,738,817 and U.S. Pat. No. 4,790,881 (all to Warner Lambert). These disclose specific constructions of capsules made of gelatin, starch and other polymers, and methods of making them by injection molding of hydrophilic polymer—water mixtures. U.S. Pat. No. 4,576,284 specifically discloses such capsules provided with a cap which closes the capsule, and which is formed in situ on the filled capsule by molding. U.S. Pat. No. 4,738,724 discloses a wide range of rigid capsule shapes and parts.

Multi-compartment capsules, including those of the type where each compartment has different drug release characteristics, or for example, contains a different drug substance or formulation are also known, for example in U.S. Pat. No. 4,738,724 (Warner-Lambert); U.S. Pat. No. 5,672,359 (University of Kentucky); U.S. Pat. No. 5,443,461 (Alza Corp.); WO 95/16438 (Cortecs Ltd.); WO 90/12567 (Helminthology Inst.); DE-A-3727894, and BE 900950 (Warner Lambert); FR 2524311, and NL 7610038 (Tapanhony N V); FR 1,454,013 (Pluripharm); U.S. Pat. No. 3,228,789 (Glassman); and U.S. Pat. No. 3,186,910 (Glassman) among others. U.S. Pat. No. 4,738,817 discloses a multicompartment capsule with a similar construction to those of U.S. Pat. No. 3,228,789 and U.S. Pat. No. 3,186,910, made of a water-plasticized gelatin. U.S. Pat. No. 4,738,817 ('817) Witter et al., U.S. Pat. No. 4,790,881 ('881), Wittwer et al., and EP 0 092 908, Wittwer, F., all discloses injection molded capsules prepared with gelatin and other excipients. Wittwer et al. '817 and '881 also prepare capsules with other hydrophilic polymers, such as hydroxypropylmethyl-cellulose phthalate (HPMCP), methylcellulose, microcrystalline cellulose, polyethylene glycol, cellulose acetate phthalate (CAP) and with polyvinylpyrrolidone.

Pharmaceutical dosage forms are also known which comprise a matrix of a solid polymer, in which a drug substance is dispersed, embedded or dissolved as a solid solution. Such matrixes may be formed by an injection molding process. This technology is discussed in Cuff G, and Raouf F, Pharmaceutical Technology, June (1998) pages 96-106. Some specific formulations for such dosage forms are disclosed in U.S. Pat. No. 4,678,516; U.S. Pat. No. 4,806,337; U.S. Pat. No. 4,764,378; U.S. Pat. No. 5,004,601; U.S. Pat. No. 5,135,752; U.S. Pat. No. 5,244,668; U.S. Pat. No. 5,139,790; U.S. Pat. No. 5,082,655; U.S. Pat. No. 5,552,159; U.S. Pat. No. 5,939,099; U.S. Pat. No. 5,741,519; U.S. Pat. No. 4,801,460; U.S. Pat. No. 6,063,821; WO 99/27909; CA 2,227,272; CA 2,188,185; CA 2,211,671; CA 2,311,308; CA 2,298,659; CA 2,264,287; CA 2,253,695; CA 2,253,700; and CA 2,257,547 among others.

U.S. Pat. No. 5,705,189, is directed to a group of co-polymers of methacrylic acid, methyl methacrylate and methyl acrylate, for use as thermoplastic agents in the production of drugs coatings, and capsules. No information is presented on the quality of the capsule formation with respect to warping or other distortions produced during or following the injection molding process,

It would be desirable to prepare a pharmaceutical dosage form in which a pharmaceutically acceptable polymeric blend is manufactured into a suitable dosage form by hot melt extrusion, or is injection molded into suitable dosage forms, which may be multicompartmental, such as in a capsule. This pharmaceutical polymeric composition as the dosage form may provide differing physio-chemical characteristics for each segment, possibly containing an active agent, In this way, a convenient dosage form can be optioned which may produce rapid, immediate, delayed, pulsatile, or modified release performance by simply selecting the appropriate polymer(s) for the manufacture of each section.

### BRIEF DESCRIPTION OF THE DRAWINGS

FIG. 1 demonstrates the dissolution profile for a capsule shell containing an active agent, having a 0.4 mm wall thickness with a 9.0 mm length by 7.7 mm diameter, under USP 2 methods, of 100 rpm in 900 ml 0.1N HCl. The dosage form component is composed of low viscosity HPC 87 w/w %, Opadry White 2 w/w %, stearyl alcohol 5 w/w %, sodium dodecyl sulphate 1 w/w % and glycerol 5 w/w %, with a Eudragit RL 100 linker.

FIG. 2 demonstrates the dissolution profile for a capsule shells 0.4 mm wall thickness with a 9.0 mm length by 7.7 mm diameter, under USP 2 methods, of 100 rpm in 900 ml 0.1N HCl. The dosage form component is composed of low viscosity HPC 83% w/w, Glycerol 5% w/w, HPMC as Pharmacoat 603 5% w/w, Sucrose Palmitate 5% w/w TiO2 1% w/w and SDS1% w/w with a Eudragit RL100 Linker.

### SUMMARY OF THE INVENTION

The present invention is directed to a novel pharmaceutical composition for making moulded articles, such as capsule shells, solid sub-units, closures or linker sub-units comprising low viscosity grade of hydroxypropylcellulose (HPC) present in an amount of about 20 to about 92% w/w; a surfactant present in an amount of about 1 to about 10% w/w; a plasticizer present in an amount of about 1% to about 20% w/w; a lubricant present in an amount of about 2% to about 15% w/w; at least one dissolution modifying excipient selected from a disintegrant, a soluble solid, a wicking agent or a water soluble filler, or a combination or mixture thereof and wherein if the disintegrant is present it is in an amount of about 2% to about 20% w/w, and wherein if the swellable solid is present it is in an amount of about 10 to about 60% w/w, and wherein if a wicking agent is present it is in an amount of about 2.5 to about 15% w/w, and if a water soluble filler is present it is in an amount of about 5 to about 10% w/w; and optionally other pharmaceutically acceptable excipients.

The present invention is also directed to the process of making the capsule shells, solid sub-units, closures or linker sub-units composed of the above formulation, and multi-component dosage forms composed of these assembled subunits, or other subunits of suitable formulations thereof.

### DETAILED DESCRIPTION OF THE INVENTION

The present invention provides for novel pharmaceutical compositions, their use in hot melt extrusion technologies, and in the making of injection molded capsule shells, linkers, spacers, multicomponent injection molded capsule shells, linkers or spacers, multicomponent pharmaceutical dosage forms, and other aspects as defined in the claims and description of this application.

Another embodiment of the invention is to provide an alternative and improved pharmaceutical dosage form which provides, inter alia, greater flexibility in the dosage form adapted to a patient's specific administration requirement, using the novel formulations of pharmaceutically acceptable polymers and suitable excipients in said dosage forms.

Another embodiment of the invention is to provide a process of producing multicomponent dosage forms comprising novel pharmaceutically acceptable polymeric blends by injection molding. These multi-component dosage forms are suitable to contain a pharmaceutically acceptable active agent, or agents, for release thereby.

In accordance with the invention, a hot melt extrusion composition, and an injection molded dosage form, such as a capsule shell, a linker or other subunit is provided for comprising a pharmaceutical composition of a low viscosity grade hydroxypropylcellulose (HPC) with other pharmaceutically acceptable excipients as will be described herein. One suitable commercially available grade of a low viscosity HPC is HPC-SSL, produced by Nisso America. HPC-SSL has a viscosity of approximately 2.0 to 2.9 mPas. Other commercially available grades of HPC, include the Nisso SL (approximately 3-5.9 mPas) and Nisso L (approximately 6-10 mPas). For purposes herein a low viscosity grade of HPC is in the range of 2.0 to less than <60 mPas, thereby providing a dosage form which when tested in vitro or in vivo, provides and immediate release of the drug contents. Generally speaking the higher the viscosity of the HPC, the longer will be the release profile of the resulting injection molded component. In one embodiment the low viscosity grade of HPC is in the range of 2.0 to about 10 mPas. In another embodiment the low viscosity grade of HPC is in the range of 2.0 to about 5.9 mPas. In another embodiment the low viscosity grade of HPC is in the range of 2.0 to about 2.9 mPas. An alternative way to also look at the HPC used as the primary polymer of the formulations herein is by molecular weight of the HPC. Generally the lower the molecular weight of the HPC, the lower the viscosity of the HPC.

The capsule or linker or other subunit comprises low viscosity HPC present in a composition in an amount of about 20 to about 92% w/w. In another embodiment the amount of low viscosity HPC present in the composition is from about 45 to 92% w/w. In another embodiment the amount of low viscosity HPC present is from about 60 to about 90% w/w. In another embodiment the amount of low viscosity HPC present is from about 80 to about 90% w/w.

The HPC-SSL is formulated in combination with various other excipients to produce a formulation that can be first extruded and then if desired injection moulded into various capsule components or dosage forms. The composition will further comprise a dissolution-modifying excipient (DME) present in an amount of about 2% w/w to about 60% w/was determined by the classification of a DME as described herein; a lubricant present in an amount of about 2% to about 15% w/w; a plasticizer present in an amount from about 1% to about 20% w/w; optionally a surfactant present in an amount of about 1 to about 10% w/w; and optionally a processing agent present in an amount from about 1% to about 10% w/w; and optionally an opacifier present in an amount of about 0.2 to about 1% w/w.

Inclusion of a surfactant in the formulation has been found to decrease the time taken for molded capsule shells to dissolve during in vitro dissolution rate testing.

The molded pharmaceutical dosage forms may comprise a plurality of sub-units, each optionally being a drug substance-containing compartment. In this case, each compartment is physically separated from an adjacent compartment. The separation is by a wall or linker subunit made of a pharmaceutically acceptable polymer material which may or may not be the same composition or polymer as either capsule compartment. In the case in which at least one of the sub-units is a drug substance-containing capsule compartment its wall thickness is in the range of about 0.1-0.8 mm. In another embodiment the wall thickness is in the range of about 0.3-0.8 mm. In another embodiment the wall thickness is in the range of about 0.3-0.5 mm. The wall thickness may be tailored depending upon the properties and dissolution release profiles desired from the product. Increases in wall thickness may be necessary to reduce warping of the components, or modification of the additional excipients in addition to this may be necessary.

The multi-component dosage form of the invention affords a high degree of versatility in that it can be composed of various combinations of different subunits having different release characteristics. For example, the sub-units which are substantially immediate release may be combined with a second substantially immediate release, or a sustained release sub-unit, such as a pulsed release sub-unit. The linker subunits, or endcaps may also be of same or different release characteristics than the capsule shell components.

Other objects and advantages of the invention will be apparent from the following description.

The present invention is directed to novel compositions of a pharmaceutically acceptable polymer which is a low viscosity hydroxypropylcellulose, and various pharmaceutically acceptable excipients, which polymeric composition may be injection molded into one or more components which can optionally be utilized together, such as in a stacked or multi-component dosage form. It is recognized that the polymeric blends may be injection molded into a single component that may also contain the active agent for oral administration within the moulded component itself, or the moulded component(s) may contain the active agent within its cavities.

The present invention also relates to the application of a pharmaceutically acceptable film coating over a component comprising the novel pharmaceutically acceptable polymeric blends as described herein. The film coating may be a delayed release formulation, or a pH control formulation as are well known in the art. Suitable coatings include but are not limited to Opadry® and Eudragit L30D-55. Enteric coatings, represented by application of L30D-55 for instance, may be applied using standard equipment such as a GMP Aerocoater column coater. The component weight gain is nominally from about 3% to about 5% w/w.

One desired attribute of the pharmaceutically acceptable dosage forms herein is to provide consistent dissolution profiles in vitro and optimally in vivo. One suitable multicomponent dosage form for use herein is disclosed in WO 01/08666, now U.S. Pat. No. 7,163,693, as well as US 2006/0057201, the contents of which are incorporated by reference herein. Other suitable formulations which may be used to derive parts of a dosage form which may be used with another part of a dosage form of this invention, e.g. a capsule compartment wall, a solid sub-unit, an end cap closure or linker sub-unit, such as a generally cylindrical subunit, such as disclosed in WO 02/060385, WO 02/060384, WO 05/089726 and WO 05/009380. Other subunit dosage forms include those encompassed by WO2009/050189, WO2009/050190, WO2009/050192, WO2009/050193 the contents of which are incorporated by reference herein; and other design patents on capsule shell embodiments such as D481456, D493518, D516714, D506545, D501550 and D501549, the contents of which are incorporated by reference herein.

The parts of the dosage form of this invention, e.g. a capsule compartment wall, a generally solid sub-unit, closure or linker sub-unit, comprise a pharmaceutically acceptable polymeric blend for oral ingestion and which polymeric blend is capable of being formed into the required shape, e.g. a capsule compartment wall, a solid sub-unit, a closure end cap or a linker. A preferred method of forming the polymer material into the desired shape is via injection molding, which may be a hot or cold runner injection molding process. Suitable injection molding machines for such a process are known in the art.

The pharmaceutical dosage form may comprise a plurality of capsule compartments each bounded and physically separated from at least one adjacent compartment by a wall made of a pharmaceutically acceptable polymer material, such as described herein, adjacent compartments being connected together in the assembled dosage form, and being retained together by the connection at least prior to administration to a patient, one or more of the compartments containing a drug substance. In one embodiment the assembled dosage form has at least two subunits, including one capsule compartment and one linker subunit. In another embodiment there are at least three subunits, comprising two capsule compartments which may be linearly disposed in the assembled dosage form, e.g. in an arrangement comprising two capsule compartments and a linker subunit; or a capsule compartment and an end closure cap and a generally solid linker subunit. In another embodiment, the capsule compartments are part of an assembled dosage form which may have four or more subunits as described above. Suitably, when there are two or more capsule compartments, one of the capsule compartments may be made of a material which is a sustained release component, i.e. so that the capsule compartment wall dissolves, bursts or is otherwise breached to release its contents after a delay, e.g. when the compartment has reached the intestine. Suitably, the other of the capsule compartments may be made of a material which is an immediate release component, i.e. so that the capsule compartment wall dissolves, bursts or is otherwise breached to release its contents immediately or effectively immediately

One or more of the capsule compartments may for example be substantially cylindrical, which term includes shapes which have a circular, oval or oblate circular cross section across the longitudinal axis, and shapes which have parallel or tapering e.g. with side walls which taper conically over at least part of their extent. Such substantially cylindrical capsule compartments may be provided with connectable parts at one or both of their longitudinally disposed ends so that the assembled dosage form may also be overall of a substantially cylindrical shape.

As noted, the low viscosity HPC polymer may be blended with additional excipients which include, but are not limited to, lubricants, such as stearyl alcohol; swelling agents; surfactants, such as SDS or the Pluronic group of agents; pore-forming/channelling agents, such as lactose or PEG; and colorants or dyes.

It is recognized that the polymeric compositions are first melted via a melt extrusion process, and may also contain additional additives or excipients to assist in melt flow, strength, brittleness, flexibility, elasticity, and other molding characteristics, these additional excipients include but are not limited to, plasticizers and processing aids.

Incorporation of a surfactant into the formulation is desired to lower the surface tension of the formulation. It is believed that incorporation of the surfactant provides for improved wettability at the surface of the polymer component as exposed to the gastrointestinal fluids, and therefore may facilitate rapid and complete dissolution of the component. Incorporation of a surfactant may also affect viscosity of the formulation. The surfactant selection may be guided by HLB values but is not necessarily a useful criterion. Higher HLB surfactants are Tween® 80 (HLB=10), Pluronic F68 (HLB=28), and SDS (HLB>40); lower HLB value surfactants, such as Pluronic F92 and F127 may also be used. Pluronic, made by BASF, USA has a synonym of Poloxamer. Pluronic F68 for instance has a molecular weight of 8,400. Pluronic F127 has a molecular weight of 12,600. Pluronics are polyoxypropylene-polyoxyethylene block copolymers.

A surfactant as used herein may also be called an oligomeric surface modifier (OSM) and includes, but is not limited to: block copolymers of ethylene oxide and propylene oxide, such as the group of commercially available Pluronics®, and which are also referred to as polyoxypropylene-polyoxyethylene block copolymers; lecithin(s); sodium dioctyl sulfosuccinate, such as Aerosol OT®; sodium lauryl sulfate which may also be referred to herein as sodium dodecyl sulfate or SDS, available commercially from multiple suppliers under tradenames such as Texapon® K12; a non-ionic hydrogenated castor oil, such as Polyoxyl 40®; polysorbates such as Tween 20, 60 & 80; the sorbitan fatty acid esters, POE sorbitan esters, polyglycerol esters, mono-, di- and tri-glyceride esters, such as such as Span®, Capmul®, Sorbester®, and Triton X-200; polyethylene glycols; glyceryl monostearate; and sucrose fatty acid ester(s). Suitably, a commercial pharmaceutically acceptable grade of sucrose fatty acid esters, such as those derived from stearic acid, oleic acid, palmitic acid, and lauric acid may be obtained from Mitsubishi-Kagaku Foods, under the tradename Surfhope SE (non-ionic emulsifiers). These are all esters which are available in products having an ester composition which is comprised of various w/w % amounts of mono, di, tri- and tetra-esters.

Suitably, the formulation may optionally contain from about 1 to about 10% w/w surfactant(s), depending upon surfactant type. When the surfactant is a sucrose fatty acid ester derivative, it will be present from about 5 to about 10% w/w surfactant. In one embodiment the sucrose fatty acid ester derivative is sucrosepalmitate, sucrose stearate, or sucrose laurate. In another embodiment the sucrose fatty acid ester derivative is sucrose palmitate (e.g. Surfhope D1616). In one embodiment when the surfactant is SDS it is present in an amount of about 0.1 to 3% w/w, suitably about 1% w/w.

The oligomeric surface modifiers, if appropriately chosen, may additionally act as absorption enhancers. Suitable absorption enhancers for use herein, include but are not limited to, chitosan, lecithin, lectins, and Vitamin E-TPGS, and combinations or mixtures thereof. Suitably, these absorption enhancers are present in a range of about 1 to about 20% w/w.

Plasticizers may be employed to assist in the melting, flow and viscosity characteristics of the composition. A plasticizer may enhance the flexibility of the moulded parts and reduces the melt viscosity which then aids the extrusion and injection moulding process. Suitable plasticizers that may be employed in this invention include triethyl citrate (TEC), triacetin, tributyl citrate, acetyl tributyl citrate (ATBC), dibutyl phthalate, dibutyl sebacate (DBS), diethyl phthalate, glycerol, vinyl pyrrolidone glycol triacetate, polyethylene glycol, polyoxyethylene sorbitan monolaurate, propylene glycol, fractionated coconut oil, or castor oil; and combinations or mixtures thereof.

In one embodiment of the present invention the plasticizer glycerol is used in the composition. In one embodiment of the present invention the plasticizer triethyl citrate is used in the composition.

Suitably, the plasticizer is present in an amount of about 1 to about 8% w/w. In one embodiment of the invention the plasticizers are present in an amount from about 2.5 to about 7.5% w/w. In another embodiment the plasticizer is present in an amount from about 5% w/w. In one embodiment of the invention the plasticizer triethyl citrate is suitably present in an amount of about 2.5 to about 7% w/w and preferably about 5%. In another embodiment triacetin is present in an amount of about 5 to about 8% w/w.

In one embodiment of the invention the plasticizer glycerol is suitably present in an amount of about 2.5 to about 7% w/w, suitably about 5%.

Dissolution modifying excipients/agents are those that assist in release modification, alter the erosion and/or swelling characteristics of the subunit or dosage form. Many different classes of agents may be used, such as the known disintegrants represented by sodium starch glycollate (Explotab®), croscarmellose sodium NF (Ac-Di-Sol® produced by FMC Biopolymer), cross-linked PVP (Kollidon-CL), copovidone (Kollidon VA 64 commercially available from BASF), or pregelatizined starch, such as Starch 1500®, commercially available from Colorcon, USA.

Suitably, when a disintegrant is present in the formulation, it is in the range of about 1 to about 10% w/w. In another embodiment of the invention the disintegrant is present from about 2 to about 5% w/w.

Another class of dissolution modifying excipients for use herein are the soluble solids such as the poly(ethylene)oxides (PEO), hydroxypropylmethyl cellulose (HPMC), hydroxyethylcellulose, hydroxymethylcellulose, or addition of a differing molecular weight of hydroxypropylcellulose which has a higher molecular weight and viscosity, such as the line of Klucel® products, e.g. Klucel EF, Klucel EXF, Klucel LF grades, and mixtures of the lower molecular weights with higher molecular weight grades such as JF or GF; polyvinyl pyrrolidone (PVP, also know as Povidone, USP), primarily grades with lower K values (K12, K15, K17, K25, but also K30 to K9); and combinations or mixtures thereof. Suitably, when a soluble solid is present it is in the range of about 1 to 65% w/w.

One source of the differing molecular weight HPC is marketed by Aqualon, a division of Hercules Incorporated, as Klucel®. Klucel HPC is produced in various grades, as determined by their intended use. Suitable Klucel polymers are Klucel EF, Klucel JH, Klucel LF, and Klucel GF. Klucel E has a viscosity in the range of 150-700 (a 300-600 mPas for EF pharm/EXF Pharm), and a molecular weight of about 80,000; J has a viscosity of 150-400 and a molecular weight of about 140,000, L has a viscosity in the range of 75-150, and a molecular weight of about 95,000; and G has a viscosity in the range of 75-400, and a molecular weight of about 370,000. It should be noted that in general one of the more important factors that affect the viscosity of the HPC is the concentration of the polymer in the solution.

One commercially available HMPC, also called hypromellose, is Pharmacoat™ 603, manufactured by Shin-Etsu Chemical Company, Japan. Pharmacoat™ 603 has a substitution type of 2910 USP designation, and a labelled viscosity of 2.4 to 3.6 mPas, a moisture permeability of 207, a methoxyl content of 28.0 to 30.0%, and a hydroxypropoxyl content of 7.0-12.0% (USP). An alternative source of commercially available hypromellose having similar viscosity and substitution is the Opadry™ line of products, available from Colorcon, New Jersey, USA, or the Methocel™ line of products from Dow Chemical Company, Michigan. It is recognized that some of the Opadry™ line also includes colorants, such as titanium dioxide which may be useful herein. One embodiment of the invention is the inclusion of Opadry™ present in amounts of about 1 to about 6% w/w. One suitable grade of Opadry™ is Opadry White. Another alternative embodiment is the inclusion of HPMC in the formulation, present in an amount of about 2 to about 6% w/w, with additional inclusion of titanium dioxide separately in amounts of 0.25 to 2% w/w, suitably around 1% w/w.

Another class of suitable dissolution modifying excipients include, but are not limited to the class of wicking agents such as the low molecular weight solutes, e.g. starch, or the class of non-reducing sugars, such as xylitol, or mannitol, present in the range of about 2.5 to about 15% w/w.

Another class of suitable dissolution modifying excipients includes the water soluble fillers, such as lactose, sorbitol are suitably present in the range of about 2.5 to about 20% w/w, alternatively from about 5 to about 10% w/w.

Another class of dissolution modifying agents are the inorganic salts such as sodium chloride, present in an amount at about 5 to about 10% w/w. In one embodiment of the invention, there is at least one or more dissolution modifying excipients added to the formulation, such as a water soluble filler, e.g. lactose and a soluble solid, such as HPMC. In one embodiment, the water soluble filler is present in an amount of 10 to 20% and the HPMC present in an amount of about 2 to 6% w/w.

It is recognized that one or more classes of dissolution modifying excipients may be used together as one embodiment of the invention. It is also recognized that more than one excipient within a class of dissolution modifying excipients may be used together as one embodiment of the invention. It is also recognized that there may be more than one excipient within a class of dissolution modifying excipients and more than one class dissolution modifying excipients present herein in any combination or mixture thereof.

Additional reagents, generally classified as processing aids, include strengthening agents, such as talc. Suitably, the processing aids are present from about 0.5 to about 10% w/w. In another embodiment, the processing aids are present from about 0.5 to about 5% w/w.

In one embodiment of the invention herein, in order to produce injection moulded components for assembly, at least one lubricant is included into the formulation as being useful to facilitate release from the injection molds. An internal lubricant is one which can provide lubrication at the die wall in the extrusion process, and mould wall in the injection moulding process. In another embodiment of the invention, the molded component is non-distorted, and unwarped, suitable for prototyping pharmaceutical active agents. In another embodiment, the molded component is non-distorted, unwarped and chemically and physically stable at accelerated stability for commercial usage, as well as for prototyping pharmaceutical active agents. Suitable mould processing lubricants, or glidants for use herein, include but are not limited to stearyl alcohol, stearic acid, glyceryl monostearate (GMS), magnesium stearate, lecithin, silicon dioxide, and combinations or mixtures thereof.

One embodiment of the present invention is the use of stearic acid or stearyl alcohol as a suitable lubricant. In another embodiment is the use of stearyl alcohol. Suitably, a commercial grade of stearyl alcohol, such as Crodacol S95 (Croda Oleochemicals) is used herein. The amount of lubricant present in the formulation is from about 2 to about 15% w/w. In another embodiment the lubricant is present from about 2.5 to about 10% w/w, and in another embodiment the lubricant is present at about 5% w/w.

Suitably, the lubricant should act as a mould processing lubricant and not cause any mould distortion nor introduce any metal ion contamination.

The final products of this invention, i.e. the capsule shells, and or other components and sub-units may additionally include constructional features and/or include materials in the composition to enhance the ease with which they can be joined together, either by simple mechanical joints, or welded together. Suitable materials for assisting such function are opacifier materials such as carbon (e.g. 0.2-0.5%), iron oxides such as ferrous oxide (e.g. 0.2-0.5%), or titanium dioxide (e.g. 0.5-2%, preferably 1% w/w) which help the polymer components to form strong mechanical or welded connections. It is recognized that in some instances commercially available preparations of dissolution modifying excipients may include in them such opacifiers.

In one embodiment, low viscosity HPC is present in an amount of 70 to 90%, alternatively 85 to 90% w/w; plasticizer 2.5 to 7% w/w; lubricant 2.5 to 10% w/w; surfactant 0.1 to 3%, and HPMC from about 1 to 6% w/w.

In one embodiment, low viscosity HPC is present in an amount of 70 to 90%, alternatively 85 to 90% w/w; plasticizer 2.5-7% w/w; lubricant 2.5 to 10% w/w; surfactant 0.1 to 3%, and at least one differing molecular weight or higher viscosity HPC from about 5 to 25% w/w.

In one embodiment, low viscosity HPC is present in an amount of 70 to 90%, alternatively 85 to 90% w/w; plasticizer 2.5 to 7% w/w; lubricant 2.5 to 10% w/w; surfactant 0.1 to 3%, and a swelling agent from about 15 to 20% w/w.

In one embodiment low viscosity HPC is present in an amount of about 87% w/w; glycerol at about 5% w/w; stearyl alcohol at about 5%, SDS at about 1%, Opadry at about 2%, or alternatively each of titanium dioxide 1% w/w and HPMC at about 1% w/w.

In one embodiment low viscosity HPC is present in an amount of about 83; about 5% w/w; stearyl alcohol at about 5%, SDS at about 1%; HPMC at about 5% w/w, titanium dioxide at about 1% w/w.

In one embodiment when the dissolution modifying excipient is the soluble solid HPMC, this is present in an amount of about 1 to 6% w/w and wherein the composition further comprises an opacifier which is titanium dioxide it is present in an amount of about 0.2 to about 1% w/w. This combination may be used with a lubricant which is glycerol, stearyl alcohol, or stearic acid. Suitably this is further in combination with a surfactant. Suitably the surfactant may be SDS or a sucrose fatty acid ester. In another embodiment the surfactant is SDS

For example each of a plurality of sub units, e.g. of the capsule compartments, solid sub-units, or combinations thereof may comprise the same or different polymer(s). For example each of a plurality of sub units, e.g. of capsule compartments, linker sub-units, or combinations thereof may comprise or contain the same or different drug substance. For example each sub-unit may contain the same drug substance but release the contents into the gastro-intestinal tract of the patient at a different rate, at different times after administration to the patient or at different places in the patient's gastro-intestinal system. Alternatively each sub-unit may contain a different drug substance, each of which may be released at the same or a different rate or time after administration or place in the patient's gastro-intestinal system.

For example two or more sub-units, e.g. two capsule compartments, may each contain different drug substances, and/or different drug substance formulations, and/or the same drug in different formulations, so that a combination of two or more drug substances or formulations may be administered to a patient.

The dosage form of this invention enables the assembly together of sub-units which differ in their drug content and/or drug content release characteristics to provide a dosage form tailored to specific administration requirements.

The dimensions and shape of each of the sub-units and hence of the overall assembled dosage form may be determined by the nature and quantity of the material to be contained therein and the intended mode of administration and intended recipients. For example a dosage form intended for oral administration may be of a shape and size similar to that of known capsules intended for oral administration.

The dosage form is particularly suitable for presentation as an oral dosage form containing one or more drug substances suitable for oral administration, and appears to be suitable for all types of such drug substance.

The drug substance(s) contained in any capsule compartment may be present in any suitable form, e.g. as a powder, granules, pellets, compact, microcapsules, gel, syrup or liquid provided that the capsule compartment wall material is sufficiently inert to the liquid content of the latter three forms. The contents of the compartments, e.g. drug substances, may be introduced into the compartments by standard methods such as those used conventionally for filling capsules, such as dosating pins, tamping pins, die filling or by hand.

As noted, the sub-units may differ from each other in their drug content release characteristics, and this may be achieved in various ways. For example, one or more linker sub-units and/or capsule compartments may be substantially immediate release, i.e. releasing their drug contents substantially immediately upon ingestion or on reaching the stomach. This may for example be achieved by means of the matrix polymer or the capsule compartment wall dissolving, disintegrating or otherwise being breached to release the drug content substantially immediately.

One or more solid sub-units and/or capsule compartments may be sustained-release sub-units. Preferably these are linker sub-units, as a bulk matrix of polymer is likely to dissolve or disperse more slowly to release its drug content than a thin walled capsule. Alternatively, the capsule containing compartment may be an immediate release subunit which comprises an enteric coating over the subunit.

One or more linker sub-units and/or capsule compartments may be pulsed-release sub-units, for example releasing their drug content at a specific predetermined point in a patient's gastro-intestinal system. This may be achieved by the use of polymer materials which dissolve or disperse only at defined pH environments, such as by certain Eudragit® polymers, for instance Amino Methacrylate Copolymer USP/NF (also referred to as Eudragit E100) which is acid labile, Eudragit FS30D or 4155F or hydroxypropylmethylcellulose acetate succinate (HPMC-AS).

In the above-described capsule compartment-linker-capsule compartment dosage forms, one capsule compartment may be effectively immediate release and the other may be a sustained, a delayed or a pulsed release capsule compartment. To achieve this for example, one capsule compartment may be made of polymer materials which cause the capsule compartment to release its drug content in the stomach or upper part of the digestive tract, and the linker (acting as a closure for the second compartment) and the second compartment itself may be made of materials e.g. the above described enteric polymers, which release their drug content only in the intestinal environment.

Adjustment of the time or location within the gastro-intestinal tract at which a sub-unit releases its drug substance content may be achieved by the polymer composition of the sub-unit material. For example the wall of different, e.g. adjacent, compartments or solid sub-units may be made of polymers which are different or which otherwise differ in their dissolution or disintegration characteristics so as to endow different compartments with different drug release characteristics.

For example the matrix, wall or closure material may be a polymer which dissolves or disperses at stomach pH to release the drug substance in the stomach. Alternatively the wall material of different compartments may differ so that different compartments have different release characteristics.

For example a linker or closure sub-unit or a capsule compartment may have respectively a matrix or a wall or a closure comprising an enteric polymer which dissolves or disperses at the pH of the small or large intestine to release the drug substance in the intestine. Suitable such polymers have been described above, for example, with reference to U.S. Pat. No. 5,705,189.

Additionally or alternatively the wall material may differ in thickness between compartments so that thicker walled compartments disrupt more slowly than thinner walled compartments.

Additionally or alternatively the compartment walls or the closure may be designed with areas or points of weakness which preferentially dissolve and may thereby determine the time of onset and/or rate of release of the drug substance content. For example such points of weakness may comprise holes, e.g. small holes, e.g. laser-drilled holes in the compartment wall or the closure, these holes being closed and/or covered with a film of a polymer material that dissolves at a pre-determined point in the digestive tract, for example an enteric polymer material. For example, such points of weakness may comprise thinned parts in a capsule compartment wall formed during the molding operation in which the capsule compartment is formed.

The sub-units may additionally or alternatively have surface or other constructional features that modify their drug release characteristics. For example linker sub-units may be provided with internal cavities or channels to alter the surface area. For example, the linker sub-units may be in the form of hollow cylinders, donuts, or toroids. Such shapes are known to tend towards first-order dissolution or erosion in liquid media and correspondingly to tend toward first-order release of drug content dispersed therein.

“Pharmaceutically acceptable agents” includes, but is not limited to, drugs, proteins, peptides, nucleic acids, nutritional agents, as described herein. This term includes therapeutic active agents, bioactive agents, active agents, therapeutic agents, therapeutic proteins, diagnostic agents, or drug(s) as defined herein, and follows the guidelines from the European Union Guide to Good Manufacturing Practice (GMP). Such substances are intended to furnish pharmacological activity or other direct effect in the diagnosis, cure, mitigation, treatment, or prevention of a disease or to affect the structure and function of the body. The substance may also include a diagnostic agent, such as an imaging agent and/or a radioactive labeled compound, which may be used to diagnose disease or for generating information relating to the structure and function of the gastrointestinal regions. The substances use may be in a mammal, or may be in a human. The pharmaceutical compositions described herein may optionally comprise one or more pharmaceutically acceptable active agents, bioactive agents, active agents, therapeutic agents, therapeutic proteins, diagnostic agents, or drug(s) or ingredients distributed within. Water solubility of an active agent is defined by the United States Pharmacoepia. Therefore, active agents which meet the criteria of very soluble, freely soluble, soluble and sparingly soluble as defined therein are encompassed this invention.

As used herein the terms “active agent”, “drug moiety” or “drug” are all used interchangeably. The terms “mold” and “mould” are used interchangeably herein.

Suitable drug substances can be selected from a variety of known classes of drugs including, but not limited to, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobactefial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, corticosteroids, cough suppressants (expectorants and mucolytics), diagnostic agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, phosphodiesterase inhibitors, neurokinin inhibitors, CSBP/RK/p38 inhibitors, antipsychotics, vasodilators and xanthines.

Preferred drug substances include those intended for oral administration. A description of these classes of drugs and a listing of species within each class can be found in Martindale, The Extra Pharmacopoeia, Twenty-ninth Edition, The Pharmaceutical Press, London, 1989, the disclosure of which is hereby incorporated herein by reference in its entirety. The drug substances are commercially available and/or can be prepared by techniques known in the art.

The polymeric blends can be preferably selected from known pharmaceutical polymers. The physico-chemical characteristics of these polymers, as well as the dimensions of the ultimate injection molded component, will dictate the properties of the dosage form, such as rapid dissolve, immediate release, delayed release, modified release such as sustained release, controlled release, or pulsatile release, etc.

The polymer blends are made by well-known methods for producing hot melt extrusions in which the selected ingredients are fed into a feed hopper of an extrusion machine. Suitable well known equipment is commercially available for producing a hot melt extrusion of the blends herein.

Therefore, one embodiment of the invention is a dosage form comprising at least one of:

(a) a shell including a first wall portion at least partially defining an interior space configured to hold a drug substance, the first wall portion being configured to dissolve within a gastrointestinal environment; or
(b) a linker including a second wall portion having a substantially cylindrical outer surface, the second wall portion configured to dissolve within a gastrointestinal environment;

wherein a respective one of the first or second wall portions are made from an extruded material comprising low viscosity hydroxypropylcellulose (HPC) present in an amount of about 20 to about 92% w/w; a surfactant present in an amount of about 1 to about 10% w/w; a plasticizer present in an amount of about 1% to about 20% w/w; a lubricant present in an amount of about 2% to about 15% w/w; and at least one dissolution modifying excipient selected from a disintegrant, a soluble solid, a wicking agent or a water soluble filler, and combination or mixtures thereof; and wherein if the disintegrant is present it is in an amount of about 2% to about 20% w/w, and wherein if the soluble solid is present it is in an amount of about 10 to about 60% w/w, and wherein if a wicking agent is present it is in an amount of about 2.5 to about 15% w/w, and if or a water soluble filler is present it is in an amount of about 2.5 to about 20% w/w; and optionally a processing aid and/or an opacifier.

Another embodiment of the invention is a dosage form comprising at least one subcomponent having a wall portion made from an extruded material comprising a low viscosity hydroxypropylcellulose (HPC) present in an amount of about 20 to about 92% w/w; a surfactant present in an amount of about 1 to about 10% w/w; a plasticizer present in an amount of about 1% to about 20% w/w; a lubricant present in an amount of about 2% to about 15% w/w; and at least one dissolution modifying excipient selected from a disintegrant, a soluble solid, a wicking agent or a water soluble filler, and combinations or mixtures thereof; and wherein if the disintegrant is present it is in an amount of about 2% to about 20% w/w, and wherein if the soluble solid is present it is in an amount of about 10 to about 60% w/w, and wherein if a wicking agent is present it is in an amount of about 2.5 to about 15% w/w, and if or a water soluble filler is present it is in an amount of about 2.5 to about 20% w/w; and optionally a processing aid and/or an opacifier.

Another embodiment of the invention is a dosage form comprising a wall portion configured to be dissolvable within a gastrointestinal environment, the wall portion made from an extruded material comprising a low viscosity hydroxypropylcellulose (HPC) present in an amount of about 20 to about 92% w/w; a surfactant present in an amount of about 1 to about 10% w/w; a plasticizer present in an amount of about 1% to about 20% w/w; a lubricant present in an amount of about 2% to about 15% w/w; and at least one dissolution modifying excipient selected from a disintegrant, a soluble solid, a wicking agent or a water soluble filler, and combinations or mixtures thereof; and wherein if the disintegrant is present it is in an amount of about 2% to about 20% w/w, and wherein if the soluble solid is present it is in an amount of about 10 to about 60% w/w, and wherein if a wicking agent is present it is in an amount of about 2.5 to about 15% w/w, and if or a water soluble filler is present it is in an amount of about 2.5 to about 20% w/w; and optionally a processing aid and/or an opacifier.

Another embodiment of the invention is a dosage form, comprising:

a) a capsule shell including a wall at least partially defining an interior space for retaining a drug substance and being configured to dissolve within a gastrointestinal environment; and

b) a linker including a wall having a substantially cylindrical outer surface and being configured to dissolve within a gastrointestinal environment;

wherein at least one of the capsule shell or the linker is made from an extruded material comprising a low viscosity hydroxypropylcellulose (HPC) present in an amount of about 20 to about 92% w/w; a surfactant present in an amount of about 1 to about 10% w/w; a plasticizer present in an amount of about 1% to about 20% w/w; a lubricant present in an amount of about 2% to about 15% w/w; and at least one dissolution modifying excipient selected from a disintegrant, a soluble solid, a wicking agent or a water soluble filler, or combinations or mixtures thereof; and wherein if the disintegrant is present it is in an amount of about 2% to about 20% w/w, and wherein if the soluble solid is present it is in an amount of about 10 to about 60% w/w, and wherein if a wicking agent is present it is in an amount of about 2.5 to about 15% w/w, and if or a water soluble filler is present it is in an amount of about 2.5 to about 20% w/w; and optionally a processing aid and/or an opacifier.

Another embodiment of the invention is a dosage form component configured as a hollow capsule, an end cap, or a linker, said component consisting essentially of an extruded or injection molded pharmaceutical composition comprising a low viscosity hydroxypropylcellulose (HPC) present in an amount of about 20 to about 92% w/w; a surfactant present in an amount of about 1 to about 10% w/w; a plasticizer present in an amount of about 1% to about 20% w/w; a lubricant present in an amount of about 2% to about 15% w/w; and at least one dissolution modifying excipient selected from a disintegrant, a soluble solid, a wicking agent or a water soluble filler, and combinations or mixtures thereof; and wherein if the disintegrant is present it is in an amount of about 10 to about 60% w/w, and wherein if a wicking agent is present it is in an amount of about 2.5 to about 15% w/w, and if or a water soluble filler is present it is in amount of about 2.5 to about 20% w/w; and optionally a processing aid and/or an opacifier.

### EXAMPLES

The invention will now be described by reference to the following examples, which are merely illustrative and are not to be construed as a limitation of the scope of the present invention. All temperatures are given in degrees centigrade; all solvents are highest available purity unless otherwise indicated.

The following examples are representative formulations of the invention as described herein. The various formulations have all been made using HPC-SSL.

Example 1

| | % w/w |
|---|---|
| HPC SSL | 60 |
| Klucel EF | 25 |
| Glycerol | 5 |
| Stearyl alcohol | 5 |
| Pharmacoat 603 | 5 |
| TOTAL | 100 |

Extrusion Prism 16 mm; screw speed 200 rpm, motor torque 37%, die pressure 0 bar; barrel temperatures 105, 110, 115, 125, 125° C.

Example 2

| | % w/w |
|---|---|
| HPC SSL | 85 |
| Stearyl alcohol | 5 |
| Pharmacoat 603 | 5 |
| glycerol | 5 |
| TOTAL | 100 |

Extrusion Prism 16 mm; screw speed 200 rpm, motor torque 45%, die pressure 0 bar; barrel temperatures 105, 110, 115, 125, 125° C.

Example 3

| | % w/w |
|---|---|
| HPC SSL | 70 |
| Klucel EF | 15 |
| Stearyl alcohol | 5 |
| Pharmacoat 603 | 5 |
| PEG 4000 | 5 |
| TOTAL | 100 |

Extrusion Prism 16 mm; screw speed 200 rpm, motor torque 66%, die pressure 0 bar; barrel temperatures 105, 110, 115, 125, 125° C.

Example 4

| | % w/w |
|---|---|
| HPC SSL | 85 |
| Stearyl alcohol | 5 |
| Pharmacoat 603 | 5 |
| Triethyl citrate | 5 |
| TOTAL | 100 |

Extrusion Prism 16 mm; screw speed 200 rpm, motor torque 64%, die pressure 3 bar; barrel temperatures 105, 110, 115, 125, 125° C.

Example 5

| | % w/w |
|---|---|
| HPC SSL | 85 |
| Stearyl alcohol | 5 |
| Pharmacoat 603 | 5 |
| Glycerol | 5 |
| TOTAL | 100 |

Pre-plasticization, blended in over 5 minutes, left overnight Extrusion Prism 16 mm; screw speed 200 rpm, motor torque 45%, die pressure 0 bar; barrel temperatures 105, 110, 115, 125, 125° C.

Example 6

| | % w/w |
|---|---|
| HPC SSL | 85 |
| Stearyl alcohol | 5 |
| Pharmacoat 603 | 5 |
| TEC | 5 |
| TOTAL | 100 |

Pre-plasticized overnight; Extrusion Prism 16 mm; screw speed 200 rpm, motor torque 61%, die pressure 12 bar; barrel temperatures 105, 110, 115, 125, 125° C.

Example 7

| | % w/w |
|---|---|
| HPC SSL | 85 |
| Stearyl alcohol | 5 |
| Pharmacoat 603 | 5 |
| PEG 400 | 5 |
| TOTAL | 100 |

Pre-plasticized overnight; Extrusion Prism 16 mm screw speed 200 rpm, motor torque 67%, die pressure 4 bar barrel temperatures 105, 110, 115, 125, 125° C.

Example 8

| | % w/w |
|---|---|
| HPC SSL | 65 |
| Klucel EF | 25 |
| Glycerol | 3 |
| Stearyl alcohol | 3 |
| Pharmacoat 603 | 5 |
| TOTAL | 100 |

Extrusion Prism 16 mm; screw speed 200 rpm, motor torque 59%, die pressure 0 bar; barrel temperatures 105, 110, 115, 125, 125° C.

Example 9

| | % w/w |
|---|---|
| HPC SSL | 70 |
| Klucel EF | 17 |
| Glycerol | 5 |
| Stearyl alcohol | 3 |
| Pharmacoat 603 | 5 |
| TOTAL | 100 |

Extrusion Prism 16 mm; screw speed 200 rpm, motor torque 50%, die pressure 0 bar; barrel temperatures 105, 110, 115, 125, 125° C.

Example 10

| | % w/w |
|---|---|
| HPC SSL | 85 |
| Glycerol | 5 |
| Stearyl alcohol | 5 |
| Pharmacoat 603 | 5 |
| TOTAL | 100 |

Extrusion Prism 16 mm; screw speed 200 rpm, motor torque 54%, die pressure 0 bar; barrel temperatures 105, 110, 115, 125, 125° C.

Example 11

| | % w/w |
|---|---|
| HPC SSL | 70 |
| Klucel EF | 25 |
| Glycerol | 5 |
| TOTAL | 100 |

Extrusion Prism 16 mm; screw speed 200 rpm, motor torque 74%, die pressure 0 bar; barrel temperatures 105, 110, 115, 125, 125° C.

Example 12

| | % w/w |
|---|---|
| HPC SSL | 85 |
| Glycerol | 5 |
| Stearyl alcohol | 5 |
| Pharmacoat 603 | 5 |
| TOTAL | 100 |

Extrusion Leistritz 27 mm; screw speed 200 rpm, motor torque 27%, die pressure 14 bar; barrel temperatures 90, 90, 90, 105, 105, 105, 110, 110, 110° C. Melt temp 115° C.

Example 13

| | % w/w |
|---|---|
| HPC SSL | 83 |
| Stearyl alcohol | 5 |
| Pharmacoat 603 | 5 |
| Glycerol | 5 |
| Explotab | 2 |
| TOTAL | 100 |

Moulded in continuous mode; Extrusion Prism 16 mm screw speed 200 rpm, motor torque 53%, die pressure 0 bar barrel temperatures 105, 110, 115, 125, 125° C.

Example 14

| | % w/w |
|---|---|
| HPC SSL | 81 |
| Stearyl alcohol | 5 |
| Pharmacoat 603 | 5 |
| Glycerol | 5 |
| Explotab | 4 |
| TOTAL | 100 |

Moulded in continuous mode; Extrusion Prism 16 mm screw speed 200 rpm, motor torque 51%, die pressure 0 bar barrel temperatures 105, 110, 115, 125, 125° C.

Example 15

| | % w/w |
|---|---|
| HPC SSL | 77 |
| Stearyl alcohol | 5 |
| Pharmacoat 603 | 5 |
| Glycerol | 5 |
| Explotab | 8 |
| TOTAL | 100 |

Moulded in continuous mode; Extrusion Prism 16 mm screw speed 200 rpm, motor torque 53%, die pressure 0 bar barrel temperatures 105, 110, 115, 125, 125° C.

Example 16

| | % w/w |
|---|---|
| HPC SSL | 75 |
| Lactose monohydrate | 10 |
| Stearyl alcohol | 5 |
| Pharmacoat 603 | 5 |
| Glycerol | 5 |
| TOTAL | 100 |

Extrusion Prism 16 mm; screw speed 200 rpm, motor torque 48%, die pressure 0 bar; barrel temperatures 105, 110, 115, 125, 125° C.

Example 17

| | % w/w |
|---|---|
| HPC SSL | 65 |
| Lactose monohydrate | 20 |
| Stearyl alcohol | 5 |
| Pharmacoat 603 | 5 |
| Glycerol | 5 |
| TOTAL | 100 |

Extrusion Prism 16 mm; screw speed 200 rpm, motor torque 40%, die pressure 0 bar; barrel temperatures 105, 110, 115, 125, 125° C.

Example 18

| | % w/w |
|---|---|
| HPC SSL | 85 |
| Glycerol | 5 |
| Stearyl alcohol | 5 |
| Pharmacoat 603 | 5 |
| TOTAL | 100 |

Extrusion Leistritz 27 mm screw speed 200 rpm, motor torque 46%, die pressure 38 bar barrel temperatures 75, 75, 75, 80, 80, 80, 85, 85, 85° C. Melt temp 86° C.

Example 19

| | % w/w |
|---|---|
| HPC SSL | 65 |
| Kollidon 17PF | 20 |
| Glycerol | 5 |
| Stearyl alcohol | 5 |
| Pharmacoat 603 | 5 |
| TOTAL | 100 |

Extrusion Leistritz 27 mm screw speed 200 rpm, motor torque 36%, die pressure 29 bar barrel temperatures 75, 75, 75, 80, 80, 80, 85, 85, 85° C. Melt temp 91° C.

Example 20

| | % w/w |
|---|---|
| HPC SSL | 65 |
| Kollicoat IR | 20 |
| Glycerol | 5 |
| Stearyl alcohol | 5 |
| Pharmacoat 603 | 5 |
| TOTAL | 100 |

Extrusion Prism 16 mm screw speed 200 rpm, motor torque 66%, die pressure 4 bar barrel temperatures 95, 100, 105, 110, 110° C.; Rubbery, would not mould.

Example 21

| | % w/w |
|---|---|
| HPC SSL | 85 |
| Pharmacoat 603 | 5 |
| Stearyl alcohol | 5 |
| Glycerol | 5 |
| TOTAL | 100 |

Extrusion Prism 16 mm; screw speed 200 rpm, motor torque 53%, die pressure 0 bar; barrel temperatures 100, 105, 110, 115, 120° C.

Example 22

| | % w/w |
|---|---|
| HPC SSL | 84.5 |
| Glycerol | 5 |
| Stearyl alcohol | 5 |
| Pharmacoat 603 | 5 |
| Titanium dioxide | 0.5 |
| TOTAL | 100 |

Extrusion Leistritz 27 mm screw speed 200 rpm, motor torque 44%, die pressure 31 bar barrel temperatures 75, 75, 75, 80, 80, 80, 85, 85, 85° C., Melt temp 91° C.

Example 23

| | % w/w |
|---|---|
| HPC SSL | 84 |
| Glycerol | 5 |
| Stearyl alcohol | 5 |
| Pharmacoat 603 | 5 |
| Titanium dioxide | 1 |
| TOTAL | 100 |

Extrusion Leistritz 27 mm;
screw speed 200 rpm,
motor torque 43%,
die pressure 35 bar;
barrel temperatures 75, 75, 75, 80, 80, 80, 85, 85, 85° C.

Example 24

| | % w/w |
|---|---|
| HPC SSL | 88 |
| Glycerol | 5 |
| Stearyl alcohol | 5 |
| Titanium dioxide | 1 |
| Sodium dodecyl sulphate | 1 |
| TOTAL | 100 |

Extrusion Prism 16 mm;
screw speed 200 rpm,
motor torque 54%,
die pressure 0 bar;
barrel temperatures100, 105, 110, 120, 120° C.

Example 25

| | % w/w |
|---|---|
| HPC SSL | 84 |
| Glycerol | 5 |
| Stearyl alcohol | 5 |
| Explotab | 5 |
| Titanium dioxide | 1 |
| TOTAL | 100 |

Extrusion Prism 16 mm;
screw speed 200 rpm,
motor torque 52%,
die pressure 0 bar;
barrel temperatures100, 105, 110, 120, 120° C.

Example 26

| | % w/w |
|---|---|
| HPC SSL | 84 |
| Glycerol | 5 |
| Stearyl alcohol | 5 |
| Ac-Di-Sol | 5 |
| Titanium dioxide | 1 |
| TOTAL | 100 |

Extrusion Prism 16 mm;
screw speed 200 rpm,
motor torque 52%,
die pressure 0 bar;
barrel temperatures100, 105, 110, 120, 120° C.

Example 27

| | % w/w |
|---|---|
| HPC SSL | 83 |
| Glycerol | 5 |
| Stearyl alcohol | 5 |
| Pharmacoat 603 | 5 |
| Titanium dioxide | 1 |
| Sodium dodecyl sulphate | 1 |
| TOTAL | 100 |

Continuous moulding;
Extrusion Prism 16 mm;
screw speed 200 rpm,
motor torque 46%,
die pressure 0 bar;
barrel temperatures 95, 95, 100, 105, 110° C.,

Example 28

| | % w/w |
|---|---|
| HPC SSL | 20 |
| Klucel EF | 66 |
| Glycerol | 2 |
| Stearyl alcohol | 5 |
| Pharmacoat 603 | 5 |
| Titanium dioxide | 1 |
| Sodium dodecyl sulphate | 1 |
| TOTAL | 100 |

Extrusion Prism 16 mm;
screw speed 200 rpm,
motor torque 53%,
die pressure 0 bar;
barrel temperatures 100, 105, 110, 115, 120° C.,

Example 29

| | % w/w |
|---|---|
| HPC SSL | 83 |
| Glycerol | 5 |
| Stearyl alcohol | 5 |
| Pharmacoat 603 | 5 |
| Titanium dioxide | 1 |
| Sodium dodecyl sulphate | 1 |
| TOTAL | 100 |

Extrusion Leistritz 27 mm;
screw speed 200 rpm,
motor torque 37%,
die pressure 31 bar;
barrel temperatures 75, 75, 75, 80, 80, 80, 80, 80, 80° C.
Melt temp 88° C.

Example 30

| | % w/w |
|---|---|
| HPC SSL | 78 |
| Glycerol | 5 |
| Pharmacoat 603 | 5 |
| Stearyl alcohol | 10 |
| Titanium dioxide | 1 |
| Sodium dodecyl sulphate | 1 |
| TOTAL | 100 |

Extrusion Prism 16 mm;
screw speed 200 rpm,
motor torque 55%,
die pressure 0 bar;
barrel temperatures 100, 105, 110, 115, 120° C.

Example 31

| | % w/w |
|---|---|
| HPC SSL | 78 |
| Stearyl alcohol | 10 |
| Pharmacoat 603 | 5 |
| Sodium dodecyl sulphate | 1 |
| Titanium dioxide | 1 |
| Glycerol | 5 |
| TOTAL | 100 |

Extrusion Prism 16 mm;
screw speed 200 rpm,
motor torque 43%,
die pressure 0 bar;
barrel temperatures 100, 105, 110, 115, 120° C.

Example 32

| | % w/w |
|---|---|
| HPC SSL | 83 |
| Glycerol | 5 |
| Sucrose ester | 5 |
| Pharmacoat 603 | 5 |
| Sodium dodecyl sulphate | 1 |
| Titanium dioxide | 1 |
| TOTAL | 100 |

Extrusion Prism 16 mm;
screw speed 200 rpm,
motor torque 60%,
die pressure 4 bar;
barrel temperatures 90, 95, 100, 110, 110° C.

Example 33

| | % w/w |
|---|---|
| HPC SSL | 76 |
| Glycerol | 7 |
| Sucrose palmitate D1616 | 10 |
| Pharmacoat 603 | 5 |
| Titanium dioxide | 1 |
| Sodium dodecyl sulphate | 1 |
| TOTAL | 100 |

Extrusion Prism 16 mm;
screw speed 200 rpm,
motor torque 33%,
die pressure 0 bar;
barrel temperatures 100, 105, 110, 115, 120° C.

Example 34

| | % w/w |
|---|---|
| HPC SSL | 76 |
| Glycerol | 7 |
| Stearyl alcohol | 2 |
| Sucrose palmitate D1616 | 8 |
| Pharmacoat 603 | 5 |
| Titanium dioxide | 1 |
| Sodium dodecyl sulphate | 1 |
| TOTAL | 100 |

Extrusion Prism 16 mm;
screw speed 200 rpm,
motor torque 33%,
die pressure 0 bar;
barrel temperatures 100, 105, 110, 115, 120° C.

Example 35

| | % w/w |
|---|---|
| HPC SSL | 88 |
| Glycerol | 5 |
| Stearic acid | 5 |
| Titanium dioxide | 1 |
| Sodium dodecyl sulphate | 1 |
| TOTAL | 100 |

Extrusion Prism 16 mm;
screw speed 200 rpm,
motor torque 66%,
die pressure 0 bar;
barrel temperatures 100, 105, 110, 115, 120° C.

Example 36

| | % w/w |
|---|---|
| HPC SSL | 83 |
| Glycerol | 5 |
| Stearic acid | 5 |
| Titanium dioxide | 1 |
| Sodium dodecyl sulphate | 1 |
| Pharmacoat 603 | 5 |
| TOTAL | 100 |

Extrusion Prism 16 mm;
screw speed 200 rpm,
motor torque 69%,
die pressure 0 bar;
barrel temperatures 100, 105, 110, 115, 120° C.

Example 37

| | % w/w |
|---|---|
| HPC SSL | 89 |
| Glycerol | 5 |
| Stearic acid | 5 |
| Titanium dioxide | 1 |
| TOTAL | 100 |

Extrusion Prism 16 mm;
screw speed 200 rpm,
motor torque 72%,
die pressure 0 bar;
barrel temperatures 100, 105, 110, 115, 120° C.

Example 38

| | % w/w |
|---|---|
| HPC SSL | 83 |
| Glycerol | 5 |
| Stearyl alcohol | 5 |
| Titanium dioxide | 1 |
| Sodium dodecyl sulphate | 1 |
| Kollidon CL | 5 |
| TOTAL | 100 |

Extrusion Prism 16 mm; screw speed 200 rpm, motor torque 69%, die pressure 0 bar; barrel temperatures 100, 105, 110, 115, 115° C.

Example 39

| | % w/w |
|---|---|
| HPC SSL | 87 |
| Glycerol | 5 |
| Stearyl alcohol | 1 |
| Titanium dioxide | 1 |
| Sodium dodecyl sulphate | 1 |
| Pharmacoat 603 | 5 |
| TOTAL | 100 |

Extrusion Prism 16 mm; screw speed 200 rpm, motor torque 75%, die pressure 0 bar; barrel temperatures 100, 105, 110, 115, 115° C.

Example 40

| | % w/w |
|---|---|
| HPC SSL | 83 |
| Glycerol | 5 |
| Stearyl alcohol | 5 |
| Titanium dioxide | 1 |
| Sodium dodecyl sulphate | 1 |
| HPMC 6 cps | 5 |
| TOTAL | 100 |

Extrusion Prism 16 mm; screw speed 200 rpm, motor torque 62%, die pressure 0 bar; barrel temperatures 100, 105, 110, 115, 115° C.

Example 41

| | % w/w |
|---|---|
| HPC SSL | 84 |
| Glycerol | 5 |
| Stearyl alcohol | 5 |
| Pharmacoat 603 | 5 |
| Sodium dodecyl sulphate | 1 |
| TOTAL | 100 |

Extrusion Prism 16 mm; screw speed 200 rpm, motor torque 60%, die pressure 0 bar; barrel temperatures 100, 105, 110, 115, 120° C.

Example 42

| | % w/w |
|---|---|
| HPC SSL | 83 |
| Glycerol | 5 |
| Stearyl alcohol | 5 |
| Pharmacoat 603 | 5 |
| Sodium dodecyl sulphate | 1 |
| Opadry OY-S_28876 | 1 |
| white | |
| TOTAL | 100 |

Extrusion Prism 16 mm; screw speed 200 rpm, motor torque 64%, die pressure 0 bar; barrel temperatures 100, 105, 110, 115, 120° C.

Example 43

| | % w/w |
|---|---|
| HPC SSL | 83 |
| Glycerol | 5 |
| Stearyl alcohol | 5 |
| Sodium dodecyl sulphate | 1 |
| Opadry OY-S white | 6 |
| TOTAL | 100 |

Extrusion Prism 16 mm; screw speed 200 rpm, motor torque 63%, die pressure 0 bar; barrel temperatures 100, 105, 110, 115, 120° C.

Example 44

| | % w/w |
|---|---|
| HPC SSL | 83 |
| Stearyl alcohol | 5 |
| Pharmacoat 603 | 5 |
| Glycerol | 5 |
| Titanium dioxide | 1 |
| Sodium dodecyl sulphate | 1 |
| TOTAL | 100 |

The excipients were pre-blended using a granulator; Extrusion-Leistritz 27 mm; screw speed 200 rpm, motor torque 35%, die pressure 25 bar; barrel temperatures 70, 70, 70, 80, 80, 80, 90, 85, 85, 85° C., melt temp 92° C.

Example 45

| | % w/w |
|---|---|
| HPC SSL | 83 |
| Glycerol | 5 |
| Sucrose ester | 5 |
| Pharmacoat 603 | 5 |
| Sodium dodecyl sulphate | 1 |
| Titanium dioxide | 1 |
| TOTAL | 100 |

The excipients were pre-blended using a granulator; Extrusion Leistritz 27 mm; screw speed 200 rpm, motor torque 35%, die pressure 25 bar; barrel temperatures 70, 70, 70, 80, 80, 80, 90, 85, 85, 85° C., melt temp 92° C.

Example 46

| | % w/w |
|---|---|
| HPC SSL | 87 |
| Glycerol | 5 |
| Stearyl alcohol | 5 |
| Sodium dodecyl sulphate | 1 |
| Opadry OY-S_28876 | 2 |
| white | |
| TOTAL | 100 |

Extrusion Prism 16 mm; screw speed 200 rpm, motor torque 59%, die pressure 0 bar; barrel temperatures 100, 105, 110, 115, 120° C.

Example 47

| | % w/w |
|---|---|
| HPC SSL | 87 |
| Stearyl alcohol | 5 |
| Glycerol | 5 |
| Opadry white | 2 |
| Sodium dodecyl sulphate | 1 |
| TOTAL | 100 |

The excipients were pre-blended using a granulator; Extrusion Leistritz 27 mm; screw speed 200 rpm, motor torque 28%, die pressure 19 bar; barrel temperatures 85, 85, 85, 90, 90, 90, 95, 95, 100° C., melt temp 92° C.

### Manufacture

The powder excipients of the above noted formulations (such as the low viscosity HPC, represented by use of HPC-SSL, stearyl alcohol and dissolution modifying polymer, etc.) were blended using a bin blender or a granulator. Any liquid excipients could be added to the blend at this stage, or later during the extrusion stage. Hot melt extrusion was generically performed on a Prism 16 mm or Leistritz 27 mm co-rotating twin-screw extruder with a temperature profile range from die to feed throat of, for example, 105-110-115-115-90-20° C. and screw speeds of 100-200 rpm. It is possible that the temperature range for the above examples may have varied by 20° C.+/−. The extruder was fed by a gravimetric powder feeder and liquid excipients could be added during the extrusion step via a peristaltic pump. The total combined feed rate was set to equal approximately 1.0 kg/hr for the 16 mm screw extruder or 10 kg/hr for the 27 mm extruder. The formulations were extruded through a 3 mm die to produce a strand that was then air cooled and then pelletized.

The pellets produced from the hot-melt extrusion process were injection moulded using an MCP 12/90 HSP mini moulder into a 9.0 mm diameter×6.9 mm height, or 7.7 diameter×9.0 mm height capsule shells, with a wall thickness of about 0.3-0.5 mm. Moulding may also have occurred on a Manner or Battenfeld system. The screw, plunger and barrel temperature was set to 130° C.-140° C. and a probe temperature to 170° C.-180° C.

When the influence of increasing the barrel temperature 5-10 degrees from the standard starting temperature of 75-85° C., and/or increasing the screw speeds from 200 rpm to 250 rpm the quality of the resulting shells was looked at with respect to its effects on shell cracking and improvements were demonstrated. Also looked at were disintegration times for the shells made from pellets at different conditions, and shells moulded from pellets with a high screw speed and barrel temperatures and these were shown to dissolve faster and more consistently than those made with sub-optimal conditions of 70(−80) degrees C. and 150 rpm, when running on the Leistritz 27 mm extruder.

### Results:

### Dissolution Analysis

Shell components were dosed with metformin or paracetamol as a soluble marker drug and sealed by clipping to an 8.35 mm diameter, 3.80 mm height injection molded linker unit. Dissolution analysis was performed via USP3 method at 10 dips per minute (dpm), for 2 hours in pH 1.2 simulated gastric fluid (SGF) followed by 6 hours in pH 6.8 simulated intestinal fluid (SIF) without sinkers.

Dissolution Testing may also be carried out using USP 2 methods at 75 rpm or 100 rpm in pH 1.2SGF using JP cage sinkers, or in 0.1 N HCl.

The following linker formulations were used in conjugation with the molded capsule shells herein:

| Linker | Ethylcellulose (N22 grade, Aqualon) | 84% (all w/w) |
|---|---|---|
| composition I: | Stearyl alcohol | 10% |
| | Glycerol | 5% |
| | BHT (butylated hydroxytoluene) | 1% |

Extrusion of this linker component was performed using a 16 mm twin-screw extruder at temperatures ranging between 120-130 degrees ° C. and samples were moulded to form linker components at temperatures between 160-180° C.

| Linker | Ammonio Methacrylate | 23% (all w/w) |
|---|---|---|
| composition II: | Copolymer Type A, USP/NF | |
| | Stearyl Alcohol | 12% |
| | HPC, such as Klucel EF | 65%. |

Extrusion of this linker component was performed using a 16 mm twin-screw extruder at temperatures ranging between 120-130 degrees ° C. and samples were moulded to form linker shaped components at temperatures between 160-180° C.

An alternative Eudragit RL-100 (Ammonio Methacrylate Copolymer Type A, USP/NF) Linker formulation for use herein is

| Ammonio Methacrylate Copolymer Type A, USP/NF | 25.00% w/w |
|---|---|
| HPC such as Klucel EF | 63.00 |
| Stearyl alcohol | 12.00 |

Extrusion/Injection moulding: Extrusion—1.2 kg/hr die temp. 110° C., 200 rpm screw, torque 35%, die pressure 1 bar; Injection Moulding—satisfactory 0.5 mm wall section shells, 180 C probe temp.

Other suitable linkers for use in the present invention include those made from HPMC-AS, such as those produced using a higher molecular weight grade of HPMC-AS HG (dissolves at 6.5-7.0), in combination with two levels of the plasticizer triacetin and the lubricant stearyl alcohol:

Content in Formulation (% w/w)

| | A | B |
|---|---|---|
| HMPC AS-HG | 90 | 85 |
| Triacetin | 5 | 10 |
| Stearyl Alcohol | 5 | 5 |

Similar formulations using HPMC-AS LG (pH 5.5) and MG (pH 5-5.6) with 5% w/w levels of triacetin have also been made. Formulations using HPMC-AS are described in McAllister et al., PCT/IB2008/003872, filed 7 Nov. 2008 or in US patent application, U.S. Ser. No. 12/266,896, the disclosure of which is incorporated by reference herein.

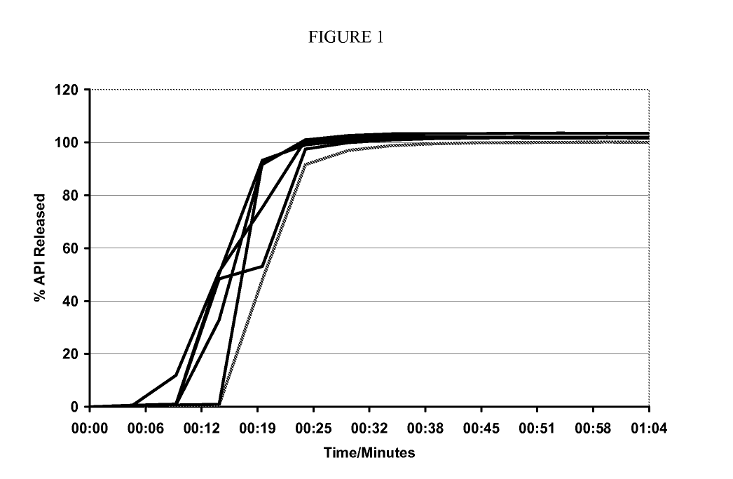

The formulation described in FIG. 1 consists of HPC SSL (87%), Opadry (2%), stearyl alcohol (5%), sodium dodecyl sulphate (1%) and glycerol (5%). The formulation has been extruded into a form suitable for injection moulding. The resulting injection molded shells produced by this formulation have been found to be pliable and have sufficient tensile strength to allow them to be attached to other linkers or shell components without breakage or deformation.

Both the extruded pellets and the moulded components show no change in physical attributes (appearance and dimensions) for at least 6 months on accelerated stability storage protocols. The pellets and dosage form components are both stable for 6 months at the following conditions: 25° C. & 60% RH, 30° C. & 65% RH, 40° C. & 75% RH. The extruded pellets and moulded components were assessed for chemical stability by assaying the content of hydroxypropoxyl groups. The hydroxypropoxyl group content following accelerated stability storage for 6 months was very similar to the group content at the initial timepoint.

Dissolution of the shell components was carried out by USP II dissolution testing in 900 mL of 0.1M HCl media at 37° C. There was complete capsule rupture observed within 10 to 15 minutes.

### Dissolution Summary Data for Other Examples:

Dissolution of the low viscosity HPC based shell components was carried out by USP II dissolution testing in 900 mL of 0.1M HCl media at 37° C. with a paddle speed of 100 rpm, or by USP III dissolution in the same media with a volume of 250 mL and a cylinder dip speed of 10 dpm.

| | Significant | Time Taken |
|---|---|---|
| | Rupture/ | to Reach |
| Formulation | Release | 80% |
| Composition | <15 | Released |
| (% w/w) | minutes | (Mean) |
| HPC SSL/Klucel EF/ | No | 26 minutes |
| Glycerol/Stearyl | | |
| Alcohol/HPMC | | |
| @60/25/5/5/5 | | |
| HPC SSL/Glycerol/ | No | 30 minutes |
| Stearyl Alcohol/ | | |
| HPMC @ 85/5/5/5 | | |
| HPC SSL/Klucel EF/ | No | 42 minutes |
| Glycerol/Stearyl | | |
| Alcohol/HPMC @ | | |
| 70/15/5/5/5 | | |
| HPC SSL/Triethyl | No | 34 minutes |
| Citrate/Stearyl | | |
| Alcohol/HPMC@ | | |
| 85/5/5/5 | | |
| HPC SSL/Glycerol/ | No | 34 minutes |
| Stearyl Alcohol/ | | |
| HPMC @ 85/5/5/5 | | |
| (Pre-blended | | |
| Glycerol) | | |
| HPC SSL/PEG 400/ | No | 22 minutes |
| Stearyl Alcohol/ | | |
| HPMC @85/5/5/5 | | |
| HPC SSL/Glycerol/ | No | 38 minutes |
| Stearyl Alcohol/ | | |
| HPMC/sodium starch | | |
| glycollate @ | | |
| 81/5/5/5/4 | | |
| HPC SSL/Glycerol/ | No | 46 minutes |
| Stearyl Alcohol/ | | |
| HPMC/sodium starch | | |
| glycollate @ | | |
| 77/5/5/5/8 | | |
| HPC SSL/Glycerol/ | No | 38 minutes |
| Stearyl Alcohol/ | | |
| HPMC/Lactose | | |
| monohydrate @ | | |
| 75/5/5/5/10 | | |
| HPC SSL/Glycerol/ | Yes | 28 minutes |
| Stearyl Alcohol/ | | |
| HPMC/Lactose | | |
| monohydrate@ | | |
| 65/5/5/5/20 | | |
| HPC SSL/Glycerol/ | Yes | 24 minutes |
| Stearyl Alcohol/ | | |
| HPMC/Titanium | | |
| Dioxide @ | | |
| 84.5/5/5/5/0.5 | | |
| HPC SSL/Glycerol/ | Yes | 26 minutes |
| Stearyl Alcohol/ | | |
| HPMC/Titanium | | |
| Dioxide @ | | |
| 84/5/5/5/1 | | |
| HPC SSL/Glycerol/ | Yes | 20 minutes |
| Stearyl Alcohol/ | | |
| HPMC/Titanium | | |
| Dioxide/Sodium | | |
| Dodecyl Sulphate@ | | |
| 83/5/5/5/1/1 | | |
| HPC SSL/Klucel EF/ | Yes | 60 minutes |
| Glycerol/Stearyl | | |
| Alcohol/HPMC/ | | |
| Titanium Dioxide/ | | |
| Sodium dodecyl | | |
| sulphate @ | | |
| 20/66/2/5/5/1/1 | | |
| HPC SSL/Glycerol/ | Yes | 26 minutes |
| HPMC/Stearyl | | |
| Alcohol/Titanium | | |
| Dioxide/Sodium | | |
| dodecyl sulphate @ | | |
| 78/5/5/10/1/1 | | |
| HPC SSL/Glycerol/ | Yes | 18 minutes |
| Sucrose palmitate/ | | |
| HPMC/Sodium | | |
| dodecyl sulphate/ | | |
| Titanium dioxide @ | | |
| 83/5/5/5/1/1 | | |
| HPC SSL/Glycerol/ | Yes | 22 minutes |
| Sucrose Palmitate/ | | |
| HPMC/Titanium | | |
| Dioxide/Sodium | | |
| dodecyl sulphate @ | | |
| 76/7/10/5/1/1 | | |
| HPC SSL/Glycerol/ | Yes | 32 minutes |
| Stearyl Alcohol/ | | |
| Sucrose palmitate/ | | |
| HPMC/Titanium | | |
| Dioxide/Sodium | | |
| dodecyl sulphate @ | | |
| 76/7/2/8/5/1/1 | | |
| HPC SSL/Glycerol/ | Yes | 18 minutes |
| Stearic Acid/ | | |
| Titanium dioxide/ | | |
| Sodium dodecyl | | |
| sulphate/HPMC @ | | |
| 83/5/5/1/1/5 | | |
| HPC SSL/Glycerol/ | Yes | 25 minutes |
| Stearyl Alcohol/ | | |
| Titanium Dioxide/ | | |
| Sodium Dodecyl | | |
| Sulphate/Kollidon | | |
| CL @ | | |
| 83/5/5/1/1/5 | | |
| HPC SSL/Glycerol/ | Yes | 18 minutes |
| Stearyl Alcohol/ | | |
| Titanium Dioxide/ | | |
| Sodium dodecyl | | |
| sulphate/HPMC @ | | |
| 87/5/1/1/1/5 | | |
| HPC SSL/Glycerol/ | Yes | 16 minutes |
| Stearyl Alcohol/ | | |
| Titanium Dioxide/ | | |
| Sodium dodecyl | | |
| sulphate/HPMC 6 | | |
| cps @ | | |
| 83/5/5/1/1/5 | | |
| HPC SSL/Glycerol/ | Yes | 24 minutes |
| Stearyl Alcohol/ | | |
| HPMC/Sodium | | |
| Dodecyl Sulphate/ | | |
| Opadry White @ | | |
| 83/5/5/5/1/1 | | |
| HPC SSL/Glycerol/ | Yes | 22 minutes |
| Stearyl Alcohol/ | | |
| Sodium Dodecyl | | |
| Sulphate/Opadry | | |
| White @ | | |
| 83/5/5/5/1/6 | | |
| HPC SSL/Stearyl | Yes | 20 minutes |
| Alcohol/Glycerol/ | | |
| Opadry White/ | | |
| Sodium dodecyl | | |
| sulphate @ | | |
| 87/5/5/2/1 | | |

A preferred use of the low viscosity HPC shells is for an immediate release dosage form. Shell rupture and initial release is best if this occurs in less than 15 minutes to ensure rapid release in the stomach, and to provide similar profiles to a conventional HPMC based capsules. The polymeric composition as a capsule shell is also desired to obtain an average of 80% of the API released no later than 30 minutes. In some instances, formulations which release as quickly as possible after initial rupture is desirable for reproducible profiles.

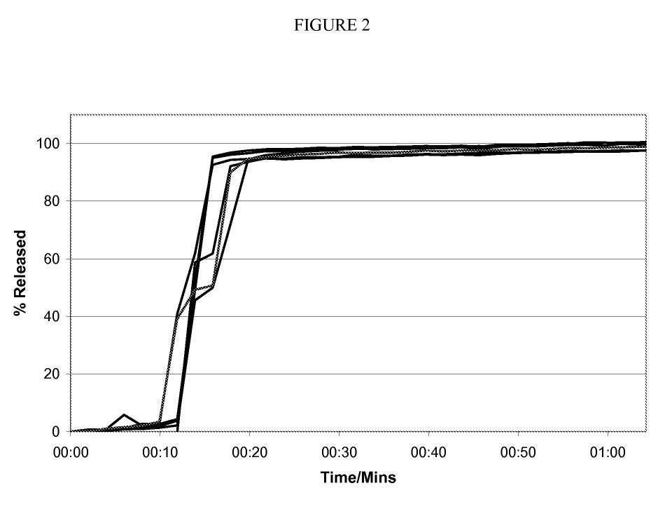

A typical release profile for formulations of the present invention are shown in FIGS. 1 and 2 herein.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the area can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A capsule comprising a shell having an outer surface and an opposed inner surface, the inner surface defining at least in part a confined space for holding a drug substance, or a generally cylindrical linker body having an outer surface, the shell or the linker being composed of an extruded material comprising a pharmaceutical composition comprising
i) a low viscosity hydroxypropylcellulose (HPC) present in an amount of about 20 to about 92% w/w;
ii) a surfactant present in an amount of about 1 to about 10% w/w;
iii) a plasticizer present in an amount of about 1% to about 20% w/w;
iv) a lubricant present in an amount of about 2% to about 15% w/w;
v) at least one dissolution modifying excipient selected from a disintegrant, a soluble solid, a wicking agent or a water soluble filler, or a combination thereof; and wherein if the disintegrant is present it is in an amount of about 2% to about 20% w/w, and wherein if the soluble solid is present it is in an amount of about 10 to about 60% w/w, and wherein if a wicking agent is present it is in an amount of about 2.5 to about 15% w/w, and if or a water soluble filler is present it is in an amount of about 2.5 to about 20% w/w, or a combination or mixture thereof; and
vi) optionally a processing aid and/or an opacifier; and
wherein the shell material between and including the inner and outer surfaces is composed of the extruded and injection molded material.

2. The shell or the linker composition according to claim 1 wherein the hydroxypropylcellulose is present in an amount of about 60 to about 90% w/w.

3. The shell or the linker composition according to claim 1 wherein the lubricant is stearyl alcohol, stearic acid, glycerol monostearate (GMS), magnesium stearate, silicon dioxide, amorphous silicic acid, or fumed silica; and combinations or mixtures thereof.

4. The shell or the linker composition according to claim 3 wherein the lubricant is stearyl alcohol or stearic acid present in an amount of about 2.5 to about 10% w/w.

5. The shell or the linker composition according to claim 4 wherein the lubricant is stearyl alcohol present in an amount of about 5% w/w.

6. The shell or the linker composition according to claim 1 wherein the at least one dissolution modifying excipient is a soluble solid.

7. The shell or the linker composition according to claim 6 wherein the soluble solid is a second hydroxypropyl cellulose having a differing molecular weight and a differing viscosity from the first low viscosity HPC, hydroxypropylmethyl cellulose, hydroxyethyl cellulose derivative, cross linked PVP, or a combination or mixture thereof.

8. The shell or the linker composition according to claim 7 wherein the soluble solid is hydroxypropylmethyl cellulose.

9. The shell or the linker composition according to claim 7 wherein the soluble solid hydroxypropylmethyl cellulose present in an amount of about 1 to 6% w/w, and further comprising an opacifier which is titanium dioxide present in an amount of about 0.2 to about 1% w/w.

10. The shell or the linker composition according to claim 6 wherein the soluble solid is one or more of hydroxypropyl cellulose polymers each having a differing molecular weight, present in a total amount of about 10% to about 66% w/w.

11. The shell or the linker composition according to claim 1 wherein the at least one dissolution modifying excipient is a soluble solid in combination with a second dissolution modifying excipient which is a water soluble filler.

12. The shell or the linker composition according to claim 11 wherein the water soluble filler is present in an amount of 10 to 20% and the soluble solid is HPMC present in an amount of about 2 to 6% w/w.

13. The shell or the linker composition according to claim 1 wherein the at least one dissolution modifying excipient is a disintegrant selected from sodium starch glycollate, croscarmellose sodium, or copovidone, or a combination or mixture thereof.

14. The shell or the linker composition according to claim 1 wherein the at least one dissolution modifying excipient is selected from polyvinyl pyrrolidone or crospovidone (cross-linked polyvinyl pyrrolidone), or a combination thereof.

15. The shell or the linker composition according to claim 1 wherein the plasticizer is triacetin, triethyl citrate (TEC), tributyl citrate, acetyl triethyl citrate (ATEC), acetyl tributyl citrate (ATBC), dibutyl phthalate, dibutyl sebacate (DBS), diethyl phthalate, vinyl pyrrolidone glycol triacetate, polyethylene glycol, glycerol, polyoxyethylene sorbitan monolaurate, propylene glycol, fractionated coconut oil or castor oil; and combinations or mixtures thereof.

16. The shell or the linker composition according to claim 15 wherein the plasticizer is triethyl citrate.

17. The shell or the linker composition according to claim 16 wherein the triethyl citrate is present in an amount of about 2.5 to about 7% w/w.

18. The shell or the linker composition according to claim 15 wherein the plasticizer is glycerol.

19. The shell or the linker composition according to claim 18 wherein the glycerol is present in an amount of about 2.5 to about 7% w/w.

20. The shell or the linker composition according to claim 1 wherein the surfactant is a sucrose fatty acid ester derivative, a block copolymer of ethylene oxide and propylene oxide, or sodium dodecyl sulfate.

21. The shell or the linker composition according to claim 20 wherein the surfactant is sodium dodecyl sulfate present in an amount of 0.1 to 3% w/w.

22. The shell or the linker composition according to claim 1 wherein the lubricant is stearyl alcohol or stearic acid, the at least one dissolution modifying excipient is HPMC, the plasticizer is TEC or glycerol, the surfactant is SDS, and optionally an opacifier which is titanium dioxide.

23. The shell or the linker composition according to claim 1 wherein the lubricant is stearyl alcohol or stearic acid, the at least one dissolution modifying excipient is HPMC, the plasticizer is glycerol, the surfactant is a sucrose fatty acid ester or SDS, and optionally an opacifier which is titanium dioxide.

24. The shell or the linker composition according to claim 1 wherein the least one dissolution modifying excipient is a soluble solid which is HPMC present in an amount of about 2 to about 10% w/w, and optionally an opacifier.

25. The shell or the linker composition according to claim 1 which is composed of any of Example 1 to 47 herein.

26. The shell or linker composition according to claim 1 which is:
i) low viscosity HPC/Stearyl Alcohol/Glycerol/Opadry White/Sodium dodecyl sulphate present in an amount of about—87/5/5/2/1% w/w; or
ii) low viscosity HPC/Stearyl Alcohol/Glycerol/HPMC/Titanium Dioxide/Sodium dodecyl sulphate present in an amount of about 83/5/5/5/1/1% w/w; or
iii) low viscosity HPC/Glycerol/Stearic Acid/Titanium Dioxide/Sodium dodecyl sulphate/HPMC present in an amount of about 83/5/5/1/1/5% w/w; or
iv) low viscosity HPC/Glycerol/Stearyl Alcohol/Titanium Dioxide/Sodium dodecyl sulphate/HPMC 6 cps present in an amount of about 83/5/5/1/1/5% w/w; or
v) low viscosity HPC/Glycerol/Stearyl Alcohol/Titanium Dioxide/Sodium Dodecyl Sulphate/Kollidon CL present in an amount of about 83/5/5/1/1/5% w/w.

27. A pharmaceutical composition comprising a
i) a low viscosity grade of hydroxypropylcellulose present in an amount of about 83 to about 87% w/w; glycerol present in an amount of about 5% w/w; stearyl alcohol or stearic acid present in an amount of about 5% w/w; sodium dodecyl sulphate present in an amount of about 1% w/w, titanium dioxide present in an amount of about 1% w/w, hydroxypropylmethylcellose (HPMC) present in an amount of about 1% w/w; or
ii) a low viscosity grade of hydroxypropylcellulose present in an amount of about 83 to about 87% w/w; glycerol present in an amount of about 5% w/w; stearyl alcohol or stearic acid present in an amount of about 5% w/w; sodium dodecyl sulphate present in an amount of about 1% w/w, and Opadry® or Opadry® white present in an amount of about 2% w/w.

28. A capsule comprising a shell having an outer surface and an opposed inner surface, the inner surface defining at least in part a confined space for holding a drug substance, or a generally cylindrical linker body having an outer surface, and the outer surface of the shell or the linker being exposed to a gastro-intestinal environment, the shell or the linker being composed of an extruded material comprising a pharmaceutical composition according to claim 27.

29. A multi-component pharmaceutical dosage form which comprises a plurality of sub-units, each sub-unit being selected from
a) a drug substance-containing capsule compartment which is soluble or disintegrable in a patient's gastro-intestinal environment for release of the drug substance contained in the capsule compartment, and
b) a linker or end cap; and
wherein the drug substance containing capsule, the linker or the end cap are comprised of a pharmaceutical composition comprising:
i) a low viscosity hydroxypropylcellulose (HPC) present in an amount of about 20 to about 92% w/w; a surfactant present in an amount of about 1 to about 10% w/w;
ii) a plasticizer present in an amount of about 1% to about 20% w/w;
iii) a lubricant present in an amount of about 2% to about 15% w/w;
iv) at least one dissolution modifying excipient selected from a disintegrant, a soluble solid, a wicking agent or a water soluble filler; and wherein if the disintegrant is present it is in an amount of about 2% to about 20% w/w, and wherein if the soluble solid is present it is in an amount of about 10 to about 60% w/w, and wherein if a wicking agent is present it is in an amount of about 2.5 to about 15% w/w, and if or a water soluble filler is present it is in an amount of about 2.5 to about 20% w/w or a combination or mixture thereof; and
v) optionally a processing aid and/or an opacifier; and
wherein the capsule compartment contains a drug substance, and in which, at least prior to administration to a patient, the sub-units are joined together in an assembled dosage form.

30. The multicomponent dosage form according to claim 29, in which at least one of the sub-units is a drug substance-containing capsule compartment having a wall with a thickness in the range of about 0.3-0.8 mm.

31. The multicomponent dosage form according to claim 29, in which at least one of the sub-units is a substantially immediate release sub-unit.

32. The multicomponent dosage form according to claim 29, in which at least one of the sub-units is a substantially sustained release sub-unit.

33. The multicomponent dosage form according to claim 29 in which the subunits are mechanically joined together.

34. The multicomponent dosage according to claim 29, wherein the composition of the drug substance containing capsule comprises:
i) a low viscosity HPC/Stearyl Alcohol/Glycerol/Opadry White/Sodium dodecyl sulphate present in an amount of about 87/5/5/2/1% w/w; or
ii) a low viscosity HPC/Stearyl Alcohol/Glycerol/HPMC/Titanium Dioxide/Sodium dodecyl sulphate present in an amount of about 83/5/5/5/1/1% w/w; or
iii) a low viscosity HPC/Glycerol/Stearic Acid/Titanium Dioxide/Sodium dodecyl sulphate/HPMC present in an amount of about 83/5/5/1/1/5% w/w; or
iv) low viscosity HPC/Glycerol/Stearyl Alcohol/Titanium Dioxide/Sodium dodecyl sulphate/HPMC 6 cps present in an amount of about 83/5/5/1/1/5% w/w; or
v) low viscosity HPC/Glycerol/Stearyl Alcohol/Titanium Dioxide/Sodium Dodecyl Sulphate/Kollidon CL present in an amount of about 83/5/5/1/1/5% w/w.

35. The multicomponent dosage form according to claim 29 wherein the linker subunit is composed of a composition comprising:
i) ethylcellulose/Stearyl alcohol/Glycerol/BHT (butylated hydroxytoluene) present in an amount of about 84/20/5/1% w/w; or
ii) Ammonio Methacrylate Copolymer Type A, USP/NF/Stearyl Alcohol/higher molecular weight HPC, such as Klucel EF present in an amount of about 23/12/65% w/w; or
iii) Ammonio Methacrylate Copolymer Type A, USP/NF/Stearyl Alcohol/higher molecular weight HPC, such as Klucel EF present in an amount of about 25/12/63% w/w.
